(19) United States
(12) Patent Application Publication
(10) Pub. No.: US 2018/0065152 A1
(43) Pub. Date: Mar. 8, 2018

(54) SEPARATING DEVICE FOR SEPARATING INSECTS FROM A REARING-BASED MATERIAL

(71) Applicant: Nordic Insect Economy Ltd, Inkeroinen (FI)

(72) Inventors: Santeri HASA, Inkeroinen (FI); Jouni SAUKKONEN, Helsinki (FI); Sami VEKKELI, Tampere (FI); Santtu VEKKELI, Inkeroinen (FI); Arttu HIRVIKORPI, Turku (FI); Rikhard HAUTALA, Turku (FI)

(21) Appl. No.: 15/258,337

(22) Filed: Sep. 7, 2016

(51) Int. Cl. B07B 1/24; B07B 1/52; A01K 67/033

(52) U.S. Cl. CPC B07B 1/24; A01K 67/033; B07B 1/522

(57) ABSTRACT

A separating device (100) for separating insects (204) from a rearing-based material (202, 203) includes a sieve part (110) and a moving part (280) for moving the sieve part in order to sieve an unseparated material (201). The unseparated material includes the insects and the rearing-based material. The sieve part includes an cylindrical sieve rotor (110) and the moving part is adapted to rotate the rotor in order to separate the insects from the rearing-based material. The rotor is inclined so that its lower end (112) is lower than its upper end (111).

### TECHNICAL FIELD

The application relates generally to a separating device for separating insects from a rearing-based material.

### BACKGROUND OF THE INVENTION

Known passive separation devices utilise passive separation based on the behavioural characteristics of reared insects.

In addition, the passive devices can use negative phototaxis in order to facilitate the separation of living insects from residue or active horizontal sieve assembly designs, e.g. shakers.

Other known devices utilise screw-type conveyors, conveyor belts, and sieve-belt based designs.

Existing separating devices suffer from insufficient ability to separate a rearing matrix material, which being liable to clogging and poor separation efficiency.

The conveyor belt-type devices are mechanically complex and, thus, more prone to breakdowns as well as being more maintenance intensive.

The horizontal vibrating sieve-type devices suffer from similar limitations to their efficacy separating the rearing matrix material and poor capacity, which hamper their operational efficiency. In addition, these devices are very noisy and demand significant occupational safety and environmental protection measures.

The passive and phototactic-type devices are slow to operate and do not facilitate the separation of the rearing matrix material from residues and waste.

### SUMMARY OF THE INVENTION

One object of the invention is to withdraw the drawbacks of existing separating devices, and provide a separating device that facilitates a recycling of a rearing matrix material and allows to separate a fine residue material efficiently from insects.

One object of the invention is fulfilled by a separating device according to the independent claim.

One embodiment of the invention is a separating device for separating insects from a rearing-based material. The device comprises a sieve part and a moving part for moving the sieve part in order to sieve an unseparated material. The unseparated material comprises the insects and the rearing-based material. The sieve part comprises an cylindrical sieve rotor and the moving part is adapted to rotate the rotor in order to separate the insects from the rearing-based material. The rotor is inclined so that its lower end is lower than its upper end.

Further embodiments of the invention are defined in the dependent claims.

### BRIEF DESCRIPTION OF THE FIGURES

The embodiments of the invention are presented with reference to the following figures:

FIG. 1 presents a separating device

FIG. 2 presents a cross-section view of the separating device

### DETAILED DESCRIPTION OF THE FIGURES

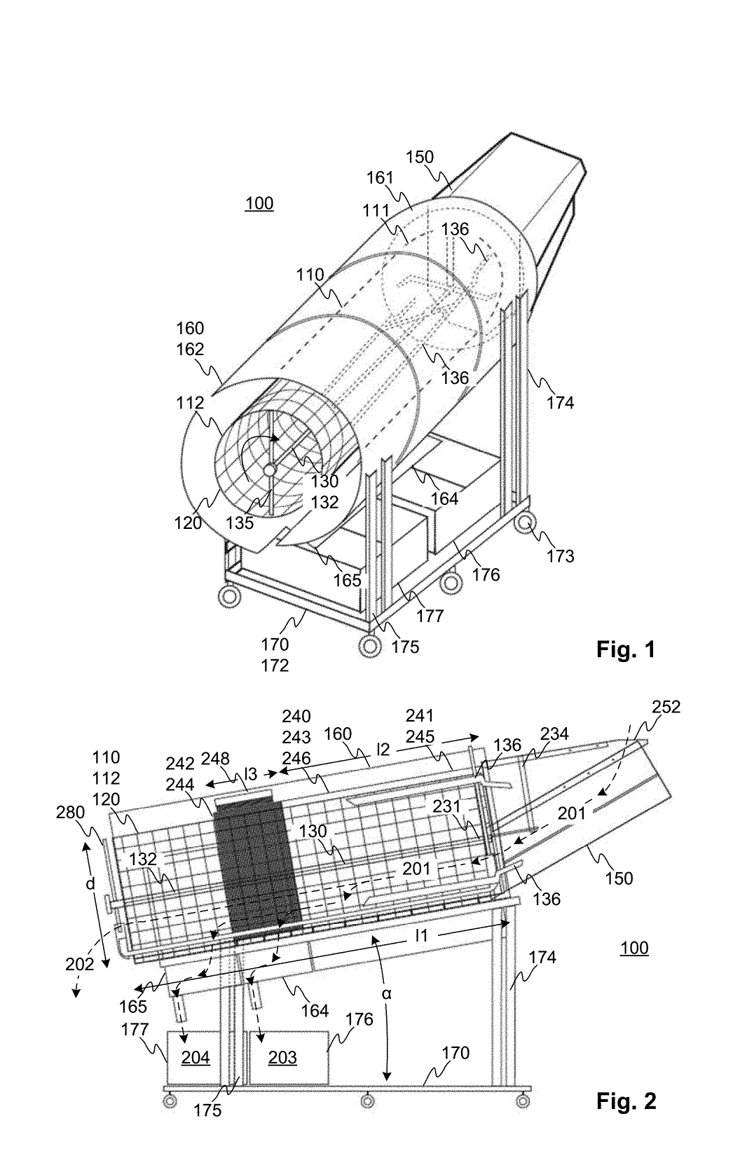

FIGS. 1 and 2 present a separating device 100 for separating insects 204 from a rearing-based material 202, 203.

The food-grade insects 204 can be maggots, e.g. mealworms, crickets, or grass-hoppers.

The unseparated material 201 can comprise the insects 204 and the material 202, 203.

The material 202, 203 can comprise a rearing matrix material 202, e.g. recyclable rearing matrices that are used for increasing a rearing surface in rearing pods. In addition, the material 202, 203 can comprise other fine material 203, e.g. residues of the insects and other fine waste.

The device 100 comprises an cylindrical sieve rotor 110 in order to sieve the unseparated material 201.

The rotor 110 has a first upper end 111 and a second lower end 112. The rotor 110 is inclined so that the lower end 112 is lower than the upper end 111.

The rotor 110 can comprise an inclined cylindrical coarse sieve 120 in order to separate the matrix material 202 from other materials 203, 204 so that the insects 204 pass through it.

The coarse sieve 120 is a coarse mesh sieve.

In addition, the rotor 110 comprise an axis 130 in order to allow the coarse sieve 120 to rotate around the axis 130.

The axis 130 has a first upper end 231 and a second lower end 132. The axis 130 is mounted inside the coarse sieve 120 so that it runs through the coarse sieve 120 according to the figures.

The upper end 231 is attached to the device 100 by means of first attachment means 234. The first means 234 allow the axis 130 to rotate.

In addition, the axis 130 is attached to the rotor 110, e.g. to its lower end 132, and/or to other parts of the device 100 by means of second attachment means 135. The second means 135 allow the axis 130 to rotate.

In addition, the rotor 110 can comprise an inclined cylindrical fine sieve 240 in order to separate the insects 204 from the fine material 203.

The fine sieve 240 has a first upper end 241 and a second lower end 242.

In addition, the fine sieve 240 is mounted so that it surrounds the coarse sieve 120 and rotates together with the coarse sieve 120 around the axis 130.

In addition, the fine sieve 240 can comprise a cylindrical solid mantle portion 243 and a cylindrical fine sieve portion 244 at its lower end 242.

The mantle portion 243 comprises the upper end 241 and a lower end 246.

The sieve portion 244 is mounted to the lower end 246 of the mantle portion 243.

In addition, the mantle portion 243 can be a plastic material and the sieve portion 244 can be a fine mesh sieve.

In addition, the device 100 can comprise at least one brush 248 in order to clean the fine sieve 240.

At least one brush 248 comprises e.g. one, two, or three separate brushes.

The brush(es) 248 is mounted on an outer surface of the sieve portion 244 so that it can dislodge the fine material 203, which is stuck in the sieve portion 244, and the insects 204, which have been seized an inner surface of the sieve portion 244, from the sieve portion 244.

In addition, the device 100 can comprise a feeder funnel part 150 in order to feed the unseparated material 201 from the rearing pods into the device 100.

The funnel part 150 is mounted to the upper end 111 of the rotor 110 so that it extends partially inside the coarse sieve 120.

In addition, the funnel part 150 has an opening 252, which allows to feed the unseparated material 201 into the device 100.

In addition, the device 100 can comprise impeller blades 136, which facilitates a gravitational movement of the unseparated material 201 from the funnel part 150 towards the coarse sieve 120.

The blades 136 are mounted to the axis 130 and inside the coarse sieve 120 so that those rotate together with the axis 130, the coarse sieve 120, and the fine sieve 240.

In addition, the blades 136 facilitate the gravitational movement of the separated matrix material 202 along an inner surface of the coarse sieve 120 towards the open lower end 112 so that the matrix material 202 can pass through the lower end 112 from the coarse sieve 120 after the coarse sieve 120 has sieved the fine material 203 and insects 204 through it.

In addition, the device 100 can comprise a cover mantle part 160 in order to cover the rotor 110.

The fixed cover 160, which surrounds the rotor 110, has an upper end 161 and an open lower end 162.

The cover 160 comprises collector parts 164, 165 in order to collect the separated fine material 203 and insects 204. The collectors 164, 165 make possible the separated material 203 and insects 204 to pass from the rotor 110 gravitationally outside the cover 160.

In addition, the device 100 can comprise a rack part 170.

The rack 170 comprises a plane part 172 in order to load necessary items 176, 177 on the rack 170, wheels 173 in order to make possible to move the device 100, and support parts 174, 175 in order to support and to incline the rotor 110.

The necessary items 176, 177 can be e.g. a collector vessel 176 for the fine material 203 and a collector vessel 177 for the insects 204.

First support parts 174 are longer than second support parts 175.

The inclination of the rotor 110 is obtained when longer support parts 174 are mounted at the upper end 161 of the cover 160 and shorter support parts 175 at its lower end 162.

The angle α of the inclination can be between 10-20°, e.g. 11, 12, 13, or 15°.

The other dimensions of the device 100 can be followings: a length I1 of the rotor 110 can be between 2000-4000 mm, e.g. 2525 mm, a length I2 of the mantle portion 243 can be between 1100-2200 mm, e.g. 1390 mm, a length I3 of the sieve portion 244 can be between 370-745 mm, e.g. 470 mm, and a diameter d of the rotor 110 can be between 650-850 mm, e.g. 749 mm.

The rack 170 facilitates a rapid change of the vessels 176, 177.

The parts 120, 130, 134, 135, 136, 243, 244, 150, 160, 170 can be made from metal and/or from composite material.

The use of metal and/or composite constructions improves hygiene and facilitates maintenance.

A mesh size in the coarse sieve 120 is larger than in the fine sieve 240. The mesh sizes can be chosen depending on specifics of the materials 202, 203, 204.

In addition, the device 100 comprises a moving part 280 in order to rotate the rotor 100 and to separate the insects 204 from the other material 202, 203.

The moving part 280 can comprise a crank or a motor, which can be mounted at the lower end 132 of the axis 130.

The crank 280 makes possible to use the device 100 manually without any electrical motor or access to electricity.

Alternatively, transmission parts of the motor can be coupled to the lower end 132 and the motor can locate e.g. in a space between the cover 160 and the lower end 112 of the coarse sieve 120, or outside the cover 160.

The device 100 is very cost-efficient, because it is simple, mechanically reliable, and it can be manufactured from comparatively cheap materials.

FIG. 2 presents how the unseparated material 201 is fed into the funnel part 150 through its opening 252.

The rotor 110 rotates before the unseparated material 201 has been fed or it starts to rotate by means of the moving part 280.

The unseparated material 201 passes gravitationally and by means of the rotating blades 136 inside the rotating coarse sieve 120.

The fine material 203 and the insects 204 pass through meshes of the coarse sieve 120 downwards, whereas the separated matrix material 202 moves gravitationally inside the coarse sieve 120 towards the open lower end 112.

The separated matrix material 202 exits gravitationally through the lower end 112 from the rotor 110 and it is collected by means of a third collector vessel, which is not presented in the figures.

This primary sieving stage provides the efficient recycling of the matrix material 202 and substantially reduces a clogging of the fine sieve 240 in subsequent stages.

The sieved fine material 203 and insects 204 moves along a space between an outer surface of the coarse sieve 120 and a solid inner surface of the rotating mantle portion 243 towards the lower ends 112, 242.

The fine material 203 passes through meshes of the rotating sieve portion 244 downwards and the separated fine material 203 exits gravitationally via collector 164 from the rotor 110, whereupon it is possible to collect the separated fine material 203 by means of the vessel 176.

On the other hand, the insects 204 moves gravitationally along a space between the outer surface of the coarse sieve 120 and an inner surface of the rotating sieve portion 244 through the sieve portion 244 towards its lower end 242.

The insects 204 exit gravitationally, after it has passed the lower end 242, via collector 165 from the rotor 110, whereupon it is possible to collect the separated insects 204 by means of the vessel 177.

The invention has been now explained above with reference to the aforementioned embodiments and the several advantages of the invention have been demonstrated.

It is clear that the invention is not only restricted to these embodiments, but comprises all possible embodiments within the scope of the following claims.

1. A separating device for separating insects from a rearing-based material, comprising:
a sieve part; and
a moving part for moving the sieve part in order to sieve an unseparated material, comprising the insects and the rearing-based material, wherein,
the sieve part comprises a cylindrical sieve rotor,
the moving part is adapted to rotate the rotor in order to separate the insects from the rearing-based material,
the rotor is inclined so that its lower end is lower than its upper end,
the rotor comprises an inclined cylindrical coarse sieve and an inclined cylindrical fine sieve,
the fine sieve is mounted so as to surround the coarse sieve and rotates together with the coarse sieve,
the fine sieve is shorter than the coarse sieve,
the coarse sieve is adapted to sieve the insects through the coarse sieve, and
the fine sieve is adapted to separate the insects from a fine residue and waste material.

2. The device of claim 1, wherein the coarse sieve is a coarse mesh sieve.

3. The device of claim 2, wherein the rotor comprises an axis for allowing the coarse sieve to rotate around the axis, and the axis is mounted to the coarse sieve so as to run through the coarse sieve.

4. The device of claim 1, further comprising a feeder funnel part for feeding the unseparated material into the device, and the feeder funnel part is mounted to the upper end of the rotor so as to extend partially inside the rotor.

5. The device of claim 1, further comprising impeller blades for facilitating a movement of the unseparated material from the funnel part towards the rotor, and the impeller blades are mounted to a rotor axis and inside the coarse sieve.

6. The device of claim 1, wherein the fine sieve is a fine mesh sieve.

7. (canceled)

8. The device of claim 6, wherein the fine sieve comprises a fine sieve portion at its lower end and a mantle portion, and the fine sieve portion is mounted to a lower end of the mantle portion.

9. The device of claim 6, further comprising at least one brush for cleaning the fine sieve, and the at least one brush is mounted on the sieve portion so as to dislodge the lodged insects and fine residue and waste material.

10. The device of claim 1, wherein the moving part comprises a motor or a crank, which is mounted at a lower end of the axis of the rotor.

11. The device of claim 1, wherein the fine sieve comprises a fine sieve portion at its lower end and a mantle portion, and the fine sieve portion is mounted to a lower end of the mantle portion.

12. The device of claim 2, further comprising a feeder funnel part for feeding the unseparated material into the device, and the feeder funnel part is mounted to the upper end of the rotor so as to extend partially inside the rotor.

13. The device of claim 3, further comprising a feeder funnel part for feeding the unseparated material into the device, and the feeder funnel part is mounted to the upper end of the rotor so as to extend partially inside the rotor.

14. The device of claim 2, further comprising impeller blades for facilitating a movement of the unseparated material from the funnel part towards the rotor, and the impeller blades are mounted to a rotor axis and inside the coarse sieve.

15. The device of claim 3, further comprising impeller blades for facilitating a movement of the unseparated material from the funnel part towards the rotor, and the impeller blades are mounted to the axis and inside the coarse sieve.

16. The device of claim 4, further comprising impeller blades for facilitating a movement of the unseparated material from the funnel part towards the rotor, and the impeller blades are mounted to a rotor axis and inside the coarse sieve.

17. The device of claim 2, wherein the fine sieve is a fine mesh sieve.

18. The device of claim 3, wherein the rotor comprises an inclined cylindrical fine sieve for separating the insects from a fine residue and waste material, and the fine sieve is a fine mesh sieve.

19. The device of claim 1, further comprising at least one brush for cleaning the fine sieve, and the at least one brush is mounted on the sieve portion so as to dislodge the lodged insects and fine residue and waste material.

20. The device of claim 8, further comprising at least one brush for cleaning the fine sieve, and the at least one brush is mounted on the sieve portion so as to dislodge the lodged insects and fine residue and waste material.
